(19) United States
(12) Patent Application Publication
(10) Pub. No.: US 2021/0300842 A1
(43) Pub. Date: Sep. 30, 2021

(54) Methods And Catalysts For Selective Olefin Isomerization

(71) Applicant: ExxonMobil Research and Engineering Company, Annandale, NJ (US)

(72) Inventors: Brandon J. O'Neill, Spring, TX (US); Joe M. Falkowski, Hampton, NJ (US); Allen W. Burton, Stewartsville, NJ (US); Scott J. Weigel, Allentown, PA (US); Randall J. Meyer, Clinton, NJ (US); Ajit B. Dandekar, Spring, TX (US)

(21) Appl. No.: 17/177,081

(22) Filed: Feb. 16, 2021

Related U.S. Application Data
(60) Provisional application No. 62/994,786, filed on Mar. 25, 2020.

(51) Int. Cl. C07C 5/27; B01J 31/16; B01J 29/70; C07C 5/03

(52) U.S. Cl. CPC C07C 5/2708; B01J 31/1691; B01J 29/70; C07C 5/03; C07C 2529/70; B01J 2231/52; B01J 2531/48; B01J 2531/004; C07C 2531/16; C07C 5/2727

(57) ABSTRACT

Zeolitic and molecular organic framework materials as catalysts suitable for generating branched olefins from linear olefins, thereby increasing the octane of a composition comprising the linear olefins. In particular, catalyst may exhibit selectivity for methyl-shift isomerization over cracking, alkylation, and oligomerization.

### FIELD

The present disclosure relates to catalysts for olefin isomerization.

### BACKGROUND

Gasoline is typically produced by cracking heavier petroleum fractions, such as vacuum gas oil, into lighter hydrocarbons suitable for use as fuels. The quality of the fuel is measured in terms of its ability to resist auto-igniting during compression. Octane is a measure of this resistance. While aromatic hydrocarbons have historically been the primary contributor to high octane, ecological implications have prompted a shift towards improving octane (or “upgrading”) with olefins and paraffins rather than using aromatics. In particular, gasoline may be upgraded by converting linear olefins to branched olefins through isomerization. For example, n-pentene may be isomerized to isopentane, which contributes more to octane rating than its linear counterpart. Isoolefins derived from n-butene and n-pentene may also be used to produce important oxygenates such as methyl tert-butyl ether and tert-amyl methyl ether, which may be added to gasoline to improve octane.

Isomerization is typically achieved by contacting linear olefins with catalytic material that facilitates rearrangement of the skeletal structure of the linear olefin, thereby creating a branched olefin. Zeolites and metal-organic framework materials are two such catalytic materials that have gained industry attention. Zeolites are more common and have been widely applied across a variety of applications. In contrast, despite their tenability and versatility in synthetic preparation, few metal-organic framework (MOF) materials exhibit catalytic activity. Only a few examples are known, such as those described in Dhakshinamoorthy, A., et al., Catal. Sci. Technol., (2012), 2, 324-330; Jiang, J., et al., J. Am. Chem. Soc. (2014), 136, 37, 12844-12847; and Sabryov, K., et al., J. Am. Chem. Soc. (2017), 139, 36, 12382-12385.

Additionally, most, if not all, catalysts that have found application in the olefin isomerization lack a desired selectivity for the methyl-shift octane-boosting reactions. In particular, many developed catalysts facilitate reactions such as double bond shift (which may be referred to as “double bond isomerization”), cracking, and molecular-weight growth reactions such as oligomerization and alkylation, all of which detract from fuel quality.

Therefore, there is a need in the art for the development of catalytic materials having enhanced selectivity for methyl-shift isomerization. Such catalysts would represent a significant benefit to the gasoline manufacturing industry.

### SUMMARY

Disclosed herein are methods for converting olefinic feeds. In one aspect, a method for converting an olefin may comprise contacting a feed comprising the olefin with a catalyst comprising metal organic framework (MOF) under conditions effective to generate a product comprising a branched isomer of the olefin, wherein not more than about 10 wt. % of the product comprises a cracking product or a molecular-weight growth reaction product, wherein a weight ratio of branched olefins in the product to molecular-weight (MW) growth reaction product in the product is greater than about 4.

In one or more embodiments, the MOF may be characterized by a plurality of Zr₆O₄(OH)₄ octahedra twelve-fold bonded together by a plurality of 4,4′-biphenyldicarboxylate linking ligands and, on average, at least one perhalo-4,4′-biphenylbisphosphonate linking ligand. Conditions effective may comprise a temperature of about 150° C. to about 300° C. and/or a pressure range of about 100 psig (689 KPa) to about 1000 psig (6.89 MPa). The product or a fraction thereof may be further processed, for example, by hydrotreating.

In another aspect, a method for converting an olefin may comprise contacting a feed comprising the olefin with a catalyst comprising a zeolite under conditions effective to generate a product comprising a branched isomer of the olefin, wherein not more than about 10 wt. % of the product comprises a cracking product or a molecular-weight growth reaction product, and wherein a weight ratio of branched olefins in the product to molecular-weight (MW) growth reaction product in the product is greater than about 4.

In one or more embodiments, the zeolite comprising pores defined by rings of 14 or more tetrahedral atoms, more preferably pores defined by rings of 21 or more tetrahedral atoms. Conditions effective may comprise a temperature of about 150° C. to about 300° C. and/or a pressure range of about 100 psig (689 KPa) to about 1000 psig (6.89 MPa).

The product or a fraction thereof may be further processed, for example, by hydrotreating.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1 provides an illustrative PXRD pattern of MOF isomerization catalyst EMM-35.

FIG. 2 provides an illustrative AT-IR spectrum of MOF isomerization catalyst EMM-35 as it compares to the AT-IR spectrum of perfluoro-4,4′-biphenylbisphosphonate.

FIG. 3 provides an illustrative ¹⁹F NMR spectrum of MOF isomerization catalyst EMM-35 having various levels of organic ligand substitution as it compares to UiO-67, and the organic ligand perfluoro-4,4′-biphenylbisphosphonate.

FIG. 4 provides example data illustrating the selectivity of EMM-35 and EMM-23 to isomerize C₆ olefins over alkylating or oligomerizing C₆ olefins.

FIG. 5 provides example data illustrating selectivity of EMM-35 and EMM-23 for isomerization over molecular-weight growth reaction at various reactor temperatures.

FIG. 6 provides example data illustrating the selectivity of EMM-35 and EMM-23 to generate branched C₆ olefins over all other competing reactions.

FIG. 7 provides example data illustrating the effect of temperature on the conversion of C₆ olefins to cracking products and/or molecular-weight growth reaction products.

### DETAILED DESCRIPTION

The present disclosure provides catalysts for the isomerization of olefins. More particularly, a feed comprising an olefin may be contacted with one or more of a zeolitic isomerization catalyst and a metal organic framework (MOF) isomerization catalyst to produce a branched isomer of the olefin.

To facilitate an understanding of the present disclosure, a number of terms and phrases are defined below.

As used herein, “zeolite” or “zeolitic” is defined to refer to a crystalline material having a porous framework structure built from tetrahedral atoms connected by bridging oxygen atoms. Examples of known zeolite frameworks are given in the “Atlas of Zeolite Frameworks” published on behalf of the Structure Commission of the International Zeolite Association”, 6^{th} revised edition, Ch. Baerlocher, L. B. McCusker, D. H. Olson, eds., Elsevier, N.Y. (2007) and the corresponding website, http://www.iza-structure.org/databases, each which is incorporated by reference herein with respect to its disclosure of zeolitic frameworks and methods for their preparation. Under this definition, a zeolite can refer to aluminosilicates having a zeolitic framework type as well as crystalline structures containing oxides of heteroatoms different from silicon and aluminum. Such heteroatoms can include any heteroatom generally known to be suitable for inclusion in a zeolitic framework, such as gallium, boron, germanium, phosphorus, zinc, antimony, tin, and/or other transition metals that can substitute for silicon and/or aluminum in a zeolitic framework. Zeolites useful in the preparation of zeolitic isomerization catalysts may be prepared from a zeolite precursor, retaining the properties described above with regards to a zeolite. Zeolites, as described herein, may be characterized by one or more of the number of tetrahedral atoms (exclusive of oxygen atoms) that define pore openings in the zeolite and an empirical formula that defines its chemical structure.

As used herein, “MOF” refers to metal-organic framework characterized by single metal ions or metal clusters linked by organic linking ligands to form one-, two-, or three-dimensional structures. MOFs are porous coordination polymers.

As used herein, the term “olefin” and grammatical derivatives thereof, refers to an unsaturated hydrocarbon chain of carbon atoms containing at least one carbon-to-carbon double bond. An olefin may be straight chain (“linear”) or branched chain (“branched”). As used herein, “branched olefin” refers to a hydrocarbon having at least one double bond wherein at least one hydrogen atom is substituted with an alkyl group.

A feed may comprise a plurality of olefins, each of which may have one or more double bonds varying in locations and total number per molecule. Branching in a bulk composition, such as a feed or product, may be characterized by branch index. Branch index within a mixture of branched olefins equals (0×% linear olefins+1×% mono-branched olefins+2×% di-branched olefins+3×% tri-branched olefins)/100; where % linear olefins+% mono-branched olefins+% di-branched olefins+% tri-branched olefins=100%. More highly branched individual olefins (e.g., tetra-branched and higher) may be weighted similarly to determine the branch index. For example, a mixture of C₆ olefins composed of 10% linear C₆, 30% mono-branched C₆, 50% di-branched C₆, and 10% tri-branched C₆ has a branch index of 1.6.

A common method for characterizing the octane number of a composition is to use

Research Octane Number (RON). As used herein, “octane number” and “RON” are used interchangeably. Although various methods are available for determining RON, in the claims below, references to Research Octane Number (RON) correspond to RON determined as described in Ghosh, P. et al. (2006) “Development of Detailed Gasoline Composition-Based Octane Model,” Ind. Eng. Chem. Res., 45(1), pp 337-345. As used herein, “high octane” is meant to describe a hydrocarbon composition having a RON of at least about 80, at least about 85, at least about 90, at least about 95, at least about 99, or about 100; or in a range of about 80 to about 100, about 90 to about 100, or about 95 to about 100.

As used herein, “methyl-shift isomerization” refers to a reaction wherein a methyl radical is removed from an olefin and reattached as a branch along the carbon backbone of the olefin molecule. While various embodiments herein are described with respect to the conversion of linear olefins to mono-branched olefins, methyl-shift isomerization may also be performed on mono-branched olefins thereby forming di-branched olefins. Similarly, two methyl radicals may be removed from an olefin and reattached as branches, thereby converting a linear olefin to a di-branched olefin.

As used herein, “cracking product” refers a hydrocarbon generated as a result of carbon-carbon bond breaking in an olefin, resulting in two molecules, each having fewer carbons than the olefin from which it was derived. Similarly, as used herein, “molecular-weight growth reaction product” refers to a hydrocarbon generated from the addition of additional atoms to an olefin, resulting in a larger molecule having more carbon atoms than either of the molecules from which it was derived. For example, alkylation and oligomerization are both considered molecular-weight growth reactions.

In one aspect, a method for converting an olefin to a branched isomer of the olefin may comprise contacting a feed comprising the olefin with a methyl-shift isomerization catalyst comprising one or more of a MOF and a zeolite under conditions effective to generate a product comprising a branched isomer of the olefin, wherein not more than 10 wt. % of the product comprises a cracking product or a molecular-weight growth reaction product. Further, the weight ratio of branched olefins to molecular-weight growth reaction product in the product is greater than about 4. Advantageously, the selectivity of the isomerization catalyst is realized it its ability to generate not more than about 10 wt. % of reaction products derived from competing side reaction (cracking, alkylation, oligomerization) and the at least four-fold proclivity for producing branched olefins over olefin oligomers and alkylation products.

In one aspect, the methyl-shift isomerization catalyst may comprise a MOF (herein referred to as a “MOF isomerization catalyst”). MOFs suitable for use in the methods disclosed herein may comprise a structure characterized by a plurality of Zr₆O₄(OH)₄ octahedra, each twelve-fold bonded to other Zr₆O₄(OH)₄ octahedra by a plurality of 4,4′-biphenyldicarboxylate linking ligands and, on average, at least one perhalo-4,4′-biphenylbisphosphonate linking ligand.

As described herein, 4,4′-biphenyldicarboxylate includes linking ligands of the structure shown as Formula I below:

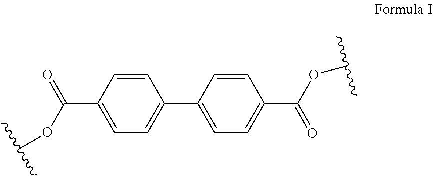 [SMILES: *OC(=O)c1ccc(-c2ccc(C(=O)O*)cc2)cc1]

As used herein, perhalo-4,4′-biphenylbisphosphonat refers to a 4,4′-biphenylbisphosphonate molecule as shown in Formula II below, wherein one or more (e.g., from 1 to 8) of R₁, R₂, R₃, R₄, R₅, R₆, R₇, and R₈ is a halogen (e.g., F, Cl, Br, and/or I):

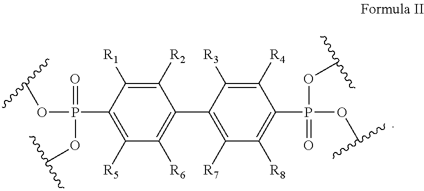

In preferred embodiments, each of R₁-R₈ is substituted with a halogen and in at least one of the substitutions, the halogen is fluorine (F). For example, in various embodiments, Formula II may be perfluoro-4,4′-biphenylbisphosphonate.

A suitable MOF may have a structure comprising a plurality of Zr₆O₄(OH)₄ octahedra bonded to adjacent Zr₆O₄(OH)₄ octahedra via twelve organic linking ligands. At least one of the linking ligands is a perhalo-4,4′-biphenylbisphosphonate linking ligand. For example, anywhere from one to twelve of the linking ligands may be a perhalo-4,4′-biphenylbisphosphonate linking ligand, from two to ten of the linking ligands may be a perhalo-4,4′-biphenylbisphosphonate linking ligand, or from four to eight of the linking ligands may be a perhalo-4,4′-biphenylbisphosphonate linking ligand. In various embodiments, the ratio of 4,4′-biphenyldicarboxylate linking ligands to perhalo-4,4′-biphenylbisphosphonate linking ligands may be from about 3:2 to about 2:3. While not wishing to be bound by theory, it is believed that the incorporating of a halogen into a linking ligand results in a lower pKa and therefore may increase catalytic activity.

In the Examples, a MOF isomerization catalyst is identified in combination with number of perhalo-4,4′-biphenylbisphosphonate linking ligand as an equivalent or “eq.” For example, the “0.4 eq.” in “EMM-35 (0.4 eq.)” means that there is about 0.4 moles of perhalo-4,4′-biphenylbisphosphonate per mole of 4,4′-biphenyldicarboxylate. The relative amount of perhalo-4,4′-biphenylbisphosphonate linking ligand can be determined through AT-IR, more specifically, by the size of the vibrational bands between 800-1200 cm⁻¹.

A suitable MOF may be prepared, for example, using UiO-67 as a template and performing ligand exchange to replace a portion of 4,4′-biphenyldicarboxylate ligands for perhalo-4,4′-biphenylbisphosphonate ligands. The exchange may be performed in a suitable solvent (e.g., dimethyl fumarate) at a temperature of about 90° C. to about 150° C., preferably about 150° C.

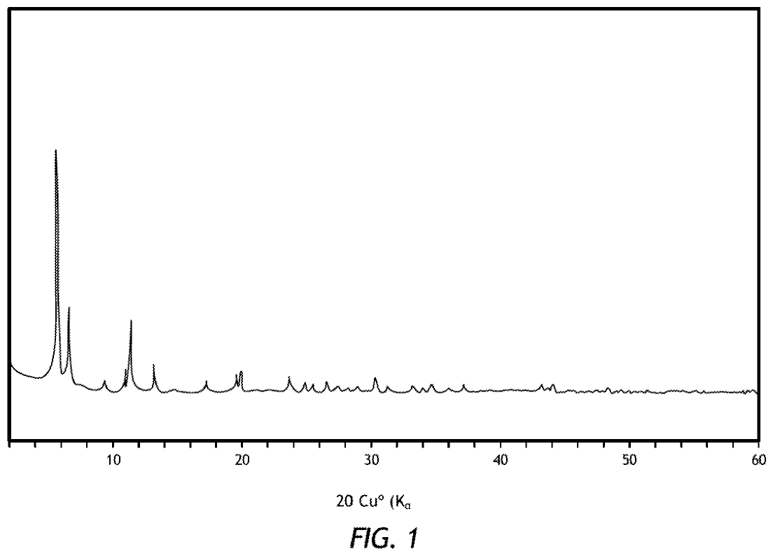

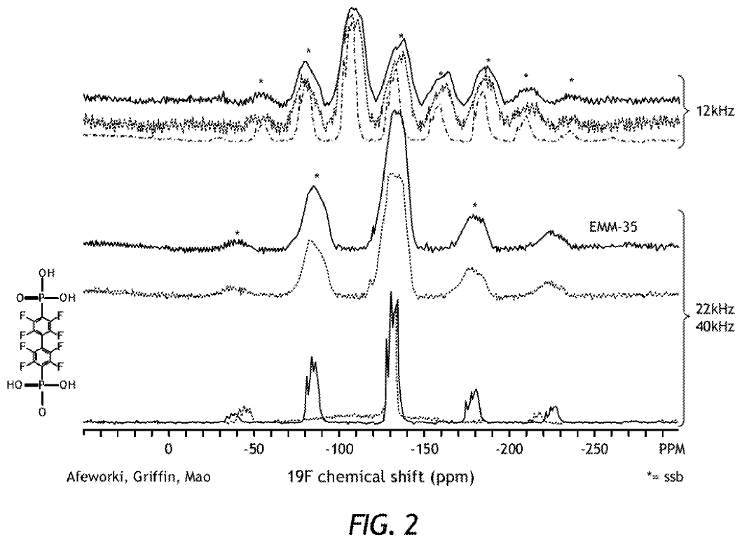

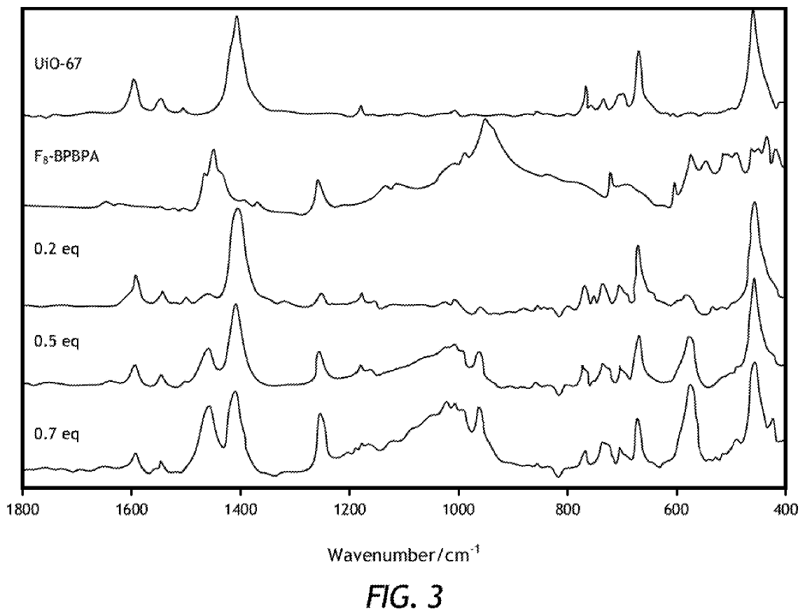

In a non-limiting example, EMM-35 developed by ExxonMobil Corporation may be a suitable MOF for use in the methods disclosed herein. EMM-35 may be characterized, for example, by one or more of the PXRD shown in FIG. 1, the ¹⁹F NMR as shown in FIG. 2, and the ATR-IR spectra shown in FIG. 3.

In another aspect, the methyl-shift isomerization catalyst may comprise a zeolite (herein referred to as a “zeolitic isomerization catalyst”). Suitable zeolites may comprise a structure characterized by pores defined by rings of 14 or more tetrahedral atoms, more preferably about 21 or more tetrahedral atoms (e.g., from 14 tetrahedral atoms to about 25 tetrahedral atoms.

A zeolitic isomerization catalyst may be employed to convert an olefin to a branched isomerization product thereof with an enhanced selectivity over generation of undesired side reaction products including, but not limited to, double-bond isomerization products, cracking products, and molecular-weight growth reaction products (e.g., alkylation products and oligomerization products). In various embodiments, a zeolitic isomerization catalyst, as disclosed herein, may be used to convert a feed comprising an olefin to a product comprising a) a branched isomer of the olefin, b) not more than about 3 wt. % of a cracking product, and c) not more than about 8 wt. % of molecular-weight growth reaction product. In more particular embodiments, a MOF isomerization catalyst may be used to generate a product comprising not more than about 1 wt. % of a cracking product and not more than about 5 wt. % of a molecular-weight growth reaction product.

Suitable zeolites may additionally be characterized by an empirical chemical formula of (X₂O₃)ₘ(YO₂)ₙ, where m is at least about 10 and n is 0 or a positive integer. For example, n and m may be (independently) 0, about 1 to about 10, about 1 to about 20, about 1 to about 30, about 1 to about 40, about 1 to about 50, about 1 to about 60, about 10 to about 20, about 10 to about 30, about 10 to about 40, about 10 to about 50, about 10 to about 60, about 20 to about 30, about 20 to about 40, about 20 to about 50, about 20 to about 60, about 30 to about 40, about 30 to about 50, about 30 to about 60, or greater than about 60. X is a trivalent element selected from one or more of B, Al, Fe, and Ga, and Y is a tetravalent element selected from one or more of Si, Ge, Sn, Ti, or Zr. In various embodiments, X is Al and Y is Si. For example, EMM-23 developed by ExxonMobil Corporation, which has been disclosed in U.S. Pat. No. 9,682,945 (herein incorporated by reference with respect to its disclosure of the synthesis and properties of EMM-23), may be a suitable zeolite for use in the methods disclosed herein. EMM-23 has a trilobed-shaped pore structure bound by 21 to 24 tetrahedral atoms. These trilobe-shaped pores of EMM-23 are intersected perpendicularly by a two-dimensional 10-atom ring channel system and have a high density of Q² and Q³ silicon species. In particular, EMM-23 may be characterized by a PXRD pattern having significant peaks in at least the following d-spacing (Å) locations: 17.5-16.3; 10.6-10.1; 9.99-9.56; and 3.766-3.704. In addition to framework alumina (e.g., X₂O₃ where X is Al), in any embodiment, a zeolite may also comprise extraframework alumina (e.g., Al-EMM-23, preparation described in Examples). EMM-23 as well as its preparation is disclosed, for example, in Willhammer, et al.; EMM-23: a stable high silica multi-dimensional zeolite with extra-large trilobe-shaped channels; J. Am. Chem. Soc., 2014, 136, 39, 13570-13573, in U.S. Pat. No. 9,205,416, and in U.S. Pat. No. 9,682,945 each of which are incorporated herein by reference with respect to their disclosure of EMM-23 properties, structure, and synthesis.

A zeolitic isomerization catalyst, in addition to comprising a zeolite as defined above, may comprise one or more impurities. These impurities may formed, for example, during the synthesis of the zeolite. Common impurities include, but are not limited to, ZSM-5 and ZSM-like zeolites, beta and beta-like zeolites, sponge-like morphologies, amorphous materials, and dense phases such as, but not limited to, quarts, tridymite, analcite, and clathrates. As such, a PXRD of an EMM-23 material containing one or more of these impurities may display baseline shift and thus may deviate somewhat from a PXRD pattern of substantially pure EMM-23. Preferably, at least about 75 wt. % of the zeolitic material in a zeolitic isomerization catalyst is EMM-23, more preferably at least about 85 wt. %, and most preferably, at least about 95 wt. %.

In any embodiment, a MOF isomerization catalyst and may comprise a MOF and optionally a binder with which the MOF may be extruded. A MOF isomerization catalyst may include from about 1 wt. % to about 99 wt. % binder, such as from about 10 wt. % to about 80 wt. %, from about 20 wt. % to about 70 wt. % or from about 30 wt. % to about 60 wt. % binder based on total weight of the total weight of the MOF isomerization catalyst. Similarly, a zeolitic isomerization catalyst may comprise a zeolite and optionally a binder with which the zeolite may be extruded. A zeolitic isomerization catalyst may include from about 1 wt. % to about 99 wt. % binder, such as from about 10 wt. % to about 80 wt. %, from about 20 wt. % to about 70 wt. % or from about 30 wt. % to about 60 wt. % binder based on total weight of the total weight of the zeolitic isomerization catalyst. Examples of suitable binders include zeolites, other inorganic materials such as clays and metal oxides such as alumina, silica, silica-alumina, titania, zirconia, iron, lanthanum, Group 1 metal oxides, Group 2 metal oxides, polymeric materials, and combinations thereof. Clays may be kaolin, bentonite, and montmorillonite and are commercially available. They may be blended with other materials such as silicates. Other suitable binders may include binary porous matrix materials (such as silica-magnesia, silica-thoria, silica-zirconia, silica-beryllia and silica-titania), and ternary materials (such as silica-alumina-magnesia, silica-alumina-thoria and silica-alumina-zirconia).

Suitable feeds include any composition comprising an olefin, preferably a linear (or normal) olefin. A feed may comprise a variety of olefins, including one or more of branched olefins, linear olefins, alpha olefins, internal olefins, dienes, and polyenes such as trienes. Olefins that may be present in a suitable feed may have at least four carbon atoms, such as from four carbon atoms to fifty or more carbon atoms. Specific examples of industrial feeds that may be converted using the methods disclosed herein include, but are not limited to, coker naphtha, cracked naphtha (including fluid catalytically cracked naphtha, hydrocracked naphtha, thermally cracked naphtha, and the like), as well as various chemical feedstocks including, but not limited to, olefins from a linear alpha olefin (LAO) synthesis, higher olefins for alcohol synthesis, product streams from alcohol upgrading, and Fisher Tropsch product streams. As such, other hydrocarbons such as, but not limited to, aromatic hydrocarbons and paraffins, may be present in a suitable feed. Zeolitic and MOF isomerization catalysts of the present disclosure may exhibit similar advantages as described above with regard to selectivity even when challenged with diverse feeds comprising paraffins and aromatics.

A feed may be contacted with one or more of a MOF isomerization catalyst and a zeolitic isomerization catalyst as described herein (e.g., a zeolite such as EMM-23) under conditions effective to convert an olefin in the feed to a branched isomer of the olefin. Conditions effective may include, for example, a pressure range of 0 psig (0 Pa) to about 3000 psig (about 20.68 MPa), such as from about 100 psig (about 103 KPa) to about 1000 psig (about 6.895 MPa), from about 100 psig (about 103 KPa) to about 500 psig (about 2.07 MPa), from about 100 psig (about 689 KPa) to about 300 psig (about 2.07 MPa) or from about 200 psig (about 1.38 MPa) to about 300 psig (about 2.07 MPa) and a temperature range of about room temperature to about 350° C., such as from about 150° C. to about 300° C., about 180° C. to about 260° C., or about 200° C. to about 240° C. Contacting may be carried out in any suitable reactor. For example, in various embodiments, a zeolitic isomerization catalyst may be contained in a fixed catalyst bed through and over which a feed may be passed.

In contacting a feed comprising an olefin with one or more of a zeolitic isomerization catalyst and a MOF isomerization catalyst as described above, a product comprising a branched isomer of the olefin may be generated. More specifically, a branched olefin having at least one more branch than the olefin from which it was derived may be generated. Thus, the weight percentage of branched olefins in product generated from contacting a feed comprising an olefin with one or more of a zeolitic isomerization catalyst and MOF isomerization catalyst may be higher than the weight percentage of branched olefins in the feed. Similarly, the branching index of a product may be higher than the branching index of the feed from which the product was derived. Surprisingly, the MOF isomerization catalysts and zeolitic isomerization catalysts described herein may display enhanced selectivity for methyl-shift isomerization over other undesirable reactions, such as double bond isomerization, which tends to be kinetically more favorable than methyl-shift isomerization. In various embodiments, one or more of a MOF isomerization catalyst and an isomerization catalyst, as disclosed herein, may be used to convert a feed comprising an olefin to a product comprising a branched isomer of the olefin at a weight percent that is at least about 4 times greater than the combined weight percent of molecular-weight growth products (e.g., alkylation products, oligomerization products). Further, not more than about 10 wt. % of the product is represented by cracking product or molecular-weight growth product. In a particular example, a product may comprise not more than about 1 wt. % of a cracking product and not more than about 5 wt. % of a molecular-weight growth reaction product.

Advantageously, the selective generation of octane-boosting branched olefins combined with the minimal generation of cracking and/or molecular-weight growth reaction products by the zeolitic isomerization catalyst and MOF isomerization catalysts disclosed herein provides a valuable route for producing a high octane product from an olefinic feed or boosting the octane rating of a lower octane composition comprising olefins.

Optionally, a product may optionally be subjected to a separation process wherein various fractions are isolated. For example, branched olefins may be isolated from other hydrocarbons such as paraffins, aromatics, and linear olefins. In another example, a linear olefin fraction may be isolated and recycled back to the feed. A product or a portion of a product may optionally be converted into a derivative product such as, but not limited to, alcohols, surfactants/emulsifiers, plasticizers, or neodecanoic acid.

The advantages may be enhanced by further processing of a product generated by the processes disclosed herein. For example, a product or a fraction of a product may be subjected to hydrotreating conditions to generate a hydrotreated product having a higher octane rating (e.g., RON) than a product generated from subjecting the initial olefinic feed to the same hydrotreating conditions.

Example Embodiments

One non-limiting embodiment of the present disclosure includes a method for converting an olefin comprising contacting a feed comprising the olefin with a catalyst comprising metal organic framework (MOF) under conditions effective to generate a product comprising a branched isomer of the olefin, wherein not more than about 10 wt. % of the product comprises a cracking product or a molecular-weight growth reaction product, wherein a weight ratio of branched olefins in the product to molecular-weight (MW) growth reaction product in the product is greater than about 4. The embodiment may further include one or more of the following Elements: Element 1: the method, wherein the MOF is characterized by a plurality of Zr₆O₄(OH)₄ octahedra twelve-fold bonded together by a plurality of 4,4′-biphenyldicarboxylate linking ligands and, on average, at least one perhalo-4,4′-biphenylbisphosphonate linking ligand; Element 2: Element 1, wherein the perhalo-4,4′-biphenylbisphosphonate linking ligand is perfluoro-4,4′-biphenylbisphosphonate linking ligand; Element 3: the method wherein the metal organic framework is EMM-3 5; Element 4: Element 1, wherein the ratio of 4,4′-biphenyldicarboxylate linking ligands to perhalo-4,4′-biphenylbisphosphonate linking ligand is from about 3:2 to about 2:3; Element 5: the method, wherein the catalyst further comprises a binder; Element 6: the method wherein the research octane number (RON) of the product is higher than the RON of the feed; Element 7: the method wherein the feed comprises one or both of coker naphtha and cracked naphtha; Element 8: the method wherein the feed further comprises one or both of a paraffin and an aromatic hydrocarbon; Element 9: the method wherein the product comprises a higher weight percentage of branched olefins than the feed; Element 10: the method wherein the conditions effective comprise a temperature of about 150° C. to about 300° C.; Element 11: the method wherein the conditions effective comprise a pressure range of about 100 psig (689 KPa) to about 1000 psig (6.89 MPa); Element 12: the method wherein the conditions effective comprise a pressure range of about 200 psig (about 1.38 MPa) to about 300 psig (about 2.07 MPa); Element 13: the method wherein at least a fraction of the product is isolated and combined with the feed; Element 14: the method, further comprising subjecting at least a portion of the product to hydrotreating conditions thereby forming a hydrotreated product having a higher octane than a product formed from subjecting the feed to the same hydrotreating conditions. Example combinations of elements include, but are not limited to, Element 1, optionally in combination with Element 2, optionally in further combination with Element 4, in further combination with one or more of Elements 3, and 5-14; Element 3 in combination with one or more of Elements 4-14; Element 5 in combination with one or more of Elements 6-14; Element 6 with one or more of Elements 7-14; Element 7 in combination with one or more of Elements 8-14; Element 8 in combination with one or more of Elements 9-14; Element 9 in combination with one or more of Elements 10-14; Element 10 in combination with one or more of Elements 11-14; Element 11 with one or more of Elements 12-14; Element 12 in combination with one or both of Elements 13 and 14; Element 13 in combination with Element 14; Element 1 and 2 in combination with Element 3; Element 1 and 2 in combination with one or both of Elements 9 and 10; and Element 1 and 2, in combination with one or both of Elements 9 and 10, and further in combination with Element 14.

Another non-limiting embodiment of the present disclosure includes a method for converting an olefin comprising contacting a feed comprising the olefin with a catalyst comprising a zeolite under conditions effective to generate a product comprising a branched isomer of the olefin, wherein not more than about 10 wt. % of the product comprises a cracking product or a molecular-weight growth reaction product, and wherein a weight ratio of branched olefins in the product to molecular-weight (MW) growth reaction product in the product is greater than about 4. The embodiment may further include any one or a combination of Elements 5-14 as well as the following Elements: Element 15: the method, wherein the catalyst comprises a zeolite comprising pores defined by rings of 14 or more tetrahedral atoms; Element 16: the method wherein the zeolite comprises pores defined by rings of 21 or more tetrahedral atoms; Element 17: the method wherein the zeolite is characterized by an empirical chemical formula of (X₂O₃)ₘ(YO₂)ₙ, wherein m is about 10 or greater, n is 0 or a positive integer, X is a trivalent element selected from one or more of B, Al, Fe, and Ga, and Y is a tetravalent element selected from one or more of Si, Ge, Sn, Ti, and Zr; Element 18: the method wherein the zeolite comprises one or both of framework alumina and extraframework alumina. Example combinations of elements include, but are not limited to, Element 5 in combination with one or more of Elements 6-18; Element 6 with one or more of Elements 7-18 Element 7 in combination with one or more of Elements 8-18; Element 8 in combination with one or more of Elements 9-18; Element 9 in combination with one or more of Elements 10-18; Element 10 in combination with one or more of Elements 11-18; Element 11 with one or more of Elements 12-18; Element 12 in combination with one or more of Elements 13-18; Element 13 in combination with one or more of Elements 14-18; Element 14 in combination with one or more of Element 15-18; Element 15 in combination with one or both of Elements 16 and 18; Element 16 in combination with Element 18; Element 15 in combination with one or both of Elements 10 and 11; and Element 16, in combination with one or both of Elements 9 and 10, and further in combination with Element 14.

To facilitate a better understanding of the embodiments of the present disclosure, the following examples of preferred or representative embodiments are given. In no way should the following examples be read to limit, or to define, the scope of the disclosure.

### EXAMPLES

### Example 1: Preparation of Al-EMM-23

133.5 g of tetramethyl orthosilicate (TMOS) was added with stirring to 462.3 g of a 15.68 wt. % solution of 1,1′-(pentane-1,5-diyl)bis(1-propylpyrrolidinium) hydroxide and 4.15 g of a 15 wt. % aluminum nitrate in a plastic beaker. The solution was covered and stirred for 3 days. After 3 days, the solution was placed in a mixer (FlackTec SpeedMixer™) and stirred for 10 minutes at 2000 rpm. The containing vessel and solution were weighed and placed in a freeze drier to remove water. After the freeze-drying, the vessel and its contents were weighed to determine mass loss. To achieve a molar ratio of H₂O:SiO₂ of 5, 8.5 g of water was added. Seeds of EMM-23 (1% on a per silica basis) were then added to the mixture, and the mixture was then placed in the mixer (FlackTec SpeedMixer™) to obtain a homogeneous gel. The gel was placed in a 300 cm³ spiral autoclave and crystallized at 150° C. for 10 days, mixing at a rate of 180 rpm. The sample was then calcined in a box furnace in a staged procedure. The sample was exposed to flowing nitrogen for two hours at room temperature, followed by a ramp from room temperature to 400° C. over a two-hour period while remaining under nitrogen flow. The temperature then remained at 400° C. for 15 minutes and then the atmosphere was switched from flowing nitrogen to flowing dried air. The temperature was then ramped from 400° C. to 540° C. over a one-hour period. The temperature remained at 540° C. for 16 hours and then the box furnace was allowed to cool to produce a solid. 20.2 g of that solid was added to an aqueous solution of 1 M aluminum nitrate (303 mL) in a 1000 mL round bottom flask and heated at 75° C. for 4 hours. The product was then recovered by filtration and washed with about 1200 mL of deionized water. The recovered solids were then dried in an oven at 95° C. overnight to yield 20.9 g of Al-EMM-23 with a chemical formula of 27SiO₂:Al₂O₃. Samples of Al-EMM-23 had a measured alpha value of 12, a surface area (BET) of 797 m²/g, and a micropore volume of 0.30 cc/g. The Al-EMM-23 crystal was extruded with alumina (Versal™ 300) at a zeolite:binder ratio of 65:35 (35 wt. % binder) into a 1/16 inch quadrulobe.

### Example 3: Preparation of High-Exchange EMM-35 (0.6 eq.)

EMM-35 was synthesized by suspending 5 grams of UiO-67 in 200 mL of dimethylsulfoxide (UiO-67 was prepared through a solvothermal synthesis of 4,4′-biphenyldicarboxylic acid and zirconyl chloride in DMF/acetic acid at 120° C. 4 grams of perfluorobiphenylbisphosphonic acid was added and the mixture stirred at 150° C. for 18 hours. Notably, it was found that increasing the temperature of the exchange from 90° C. to 150° C. resulted in materials that maintained their structure and surface area and allowed for higher degrees of ligand substitution to be achieved. The reaction was then filtered and re-suspended in dimethylsulfoxide and heated with stirring at 150° C. for an additional 18 hours. This washed material was then filtered and washed with acetone and then washed using a Soxhlet extractor with acetone for 3 hours. This material was then dried in a vacuum oven at 90° C.

### Example 4: Preparation of Low-Exchange EMM-35 (0.4 eq.)

EMM-35 was synthesized by suspending 5 grams of UiO-67 in 200 mL of dimethylsulfoxide. 2 grams of perfluorobiphenylbisphosphonic acid was added and the mixture stirred at 150° C. for 18 hours. The reaction was then filtered and re-suspended in dimethylsulfoxide and heated with stirring at 150° C. for an additional 18 hours. This washed material was then filtered and washed with acetone and then washed using a Soxhlet extractor with acetone for 3 hours. This material was then dried in a vacuum oven at 90° C.

### Example 5: Olefin Isomerization

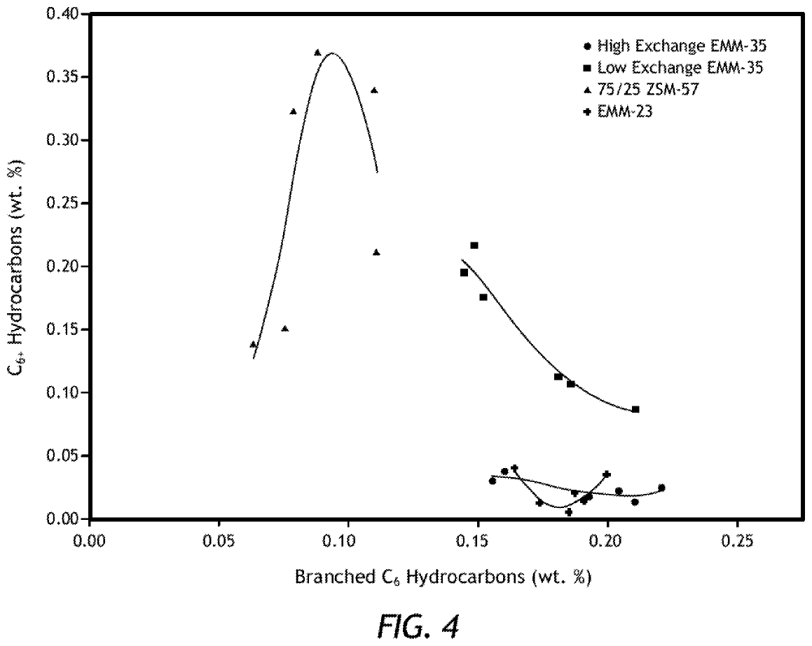

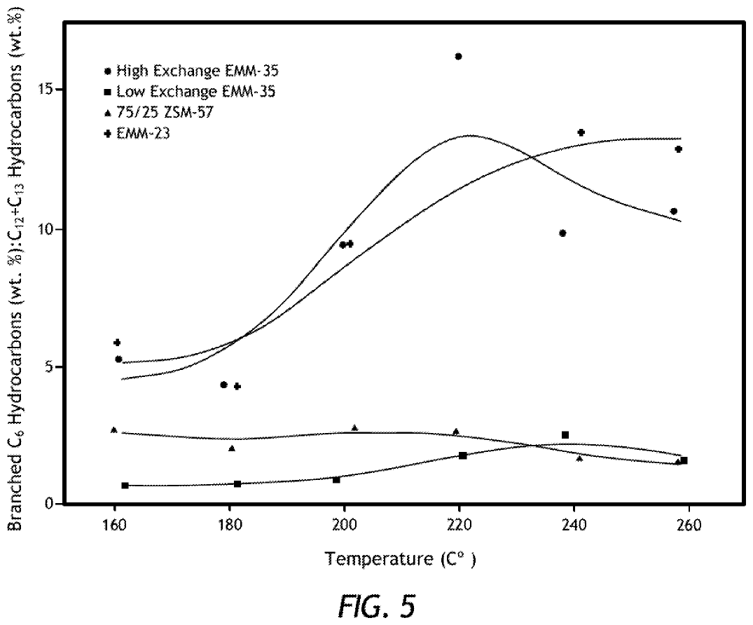

A feed comprising 26 wt. % 1-hexene, 35 wt. % heptane , and 39 w.% toluene was contacted with each of the following catalysts: EMM-35 (0.6 eq.), EMM-35 (0.4 eq.), EMM-23, and ZSM-57. The reaction was carried out at a WHSV of 2 hours⁻¹, a temperature of 160° C. to 260° C., and a pressure of 260 psig (about 1.72 MPa). Product was analyzed by gas chromatography to quantify the amount of branched C₆ hydrocarbons present as well as the amount of hydrocarbons heavier than C₆. FIG. 4 depicts the results of this analysis for each of the MOF and zeolitic isomerization catalysts tested, plotting heavy hydrocarbons, representing C₆ olefins that were alkylated or oligomerized, on the y-axis and branched C₆ hydrocarbons on the x-axis. Data sets residing high on the x-axis and low on the y-axis represent those catalysts that favor branching over molecular-weight growth (desirable), whereas those residing high on the y-axis and low on the x-axis represent catalysts favoring molecular-weight growth (undesirable). Similarly, FIG. 5 depicts a graph plotting a ratio of branched C₆ olefins to C₁₂ and C₁₃ olefins against reactor temperature. EMM-23 and EMM-35 catalysts, thus, appear to be particularly suited for selective methyl-shift isomerization over molecular-weight growth reactions. This trend becomes even more apparent at higher reactor temperatures, which is surprising since higher temperatures typically favor molecular-weight growth reactions.

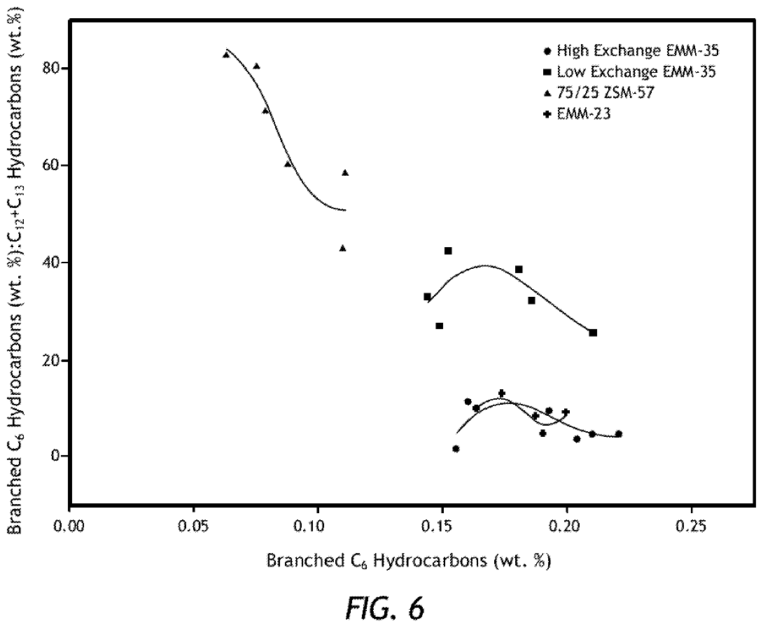

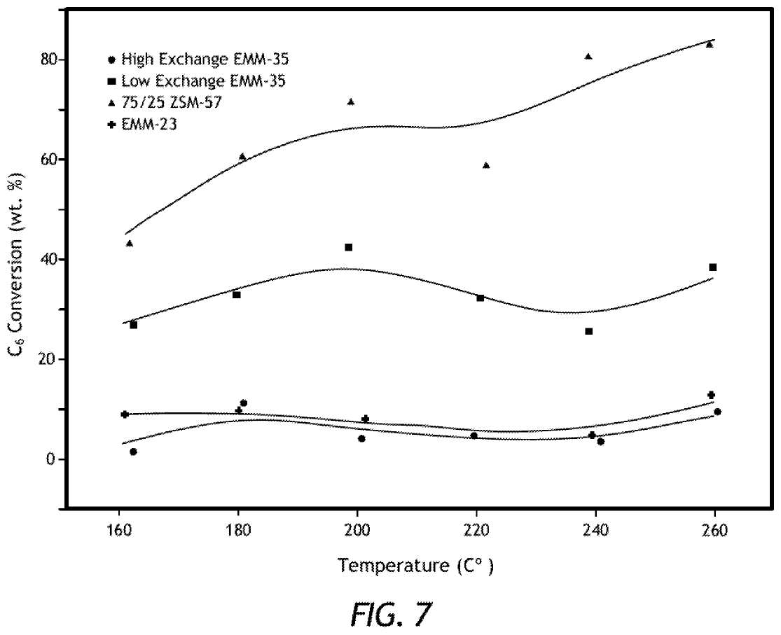

FIG. 6 depicts a graph plotting C₆ conversion on the y-axis against branched C₆ hydrocarbons on the x-axis. As used herein, “C₆ conversion” represents conversion of C₆ olefins to a hydrocarbon having more than or less than six carbons. Similarly, FIG. 7 depicts a graph plotting C₆ conversion on the y-axis against reactor temperature on the x-axis. Thus, EMM-23 and EMM-35 catalysts appear also to be particularly suited for selective methyl-shift isomerization and maintain that selectivity over both molecular-weight growth and cracking reactions over a wide range of temperatures.

### Example 6

The isomerization activity of EMM-35 and EMM-23 (Al) was analyzed by the McVicker test against UiO-67, which is a MOF isostructural with, but having a lower acidity than, EMM-35. A feed of 6.7% 2-methyl-2-pentene in helium was exposed to 0.5 g of catalyst at a feed rate of 160 sccm at 250° C. and atmospheric pressure. Table 1 below reports the initial conversion after 5 minutes on stream.

TABLE 1

| | C₁-C₅ | 4-methyl- | 3-methyl- | 2,3-dimethyl- | cis-4-methyl- |
|---|---|---|---|---|---|
| | hydrocarbons | 1-pentene | 1-pentene | 1-butene | 2-pentene |
| UiO-67 | 0 | 0 | 0 | 0 | 0 |
| EMM-35 | 0.178 | 0.135 | 0.167 | 1.119 | 2.098 |
| EMM-23 (Al) | 0.949 | 1.225 | 0.606 | 0.232 | 1.643 |

| | trans-4-methyl- | 2-methyl-1- | | 2-ethyl-1- | cis/trans-3- |
|---|---|---|---|---|---|
| | 2-pentene | pentene | 1-hexene | butene | hexene |
| UiO-67 | 0 | 1.384 | 0 | 0 | 0 |
| EMM-35 | 8.864 | 22.346 | 0 | 0.21 | 0.03 |
| EMM-23 (Al) | 9.221 | 18.497 | 0 | 0.956 | 0.294 |

| | 2-methyl-2- | cis-3-methyl- | cis-2- | trans-3-methyl- | 2,3-dimethyl- |
|---|---|---|---|---|---|
| | pentene | 2-pentene | hexene | 2-pentene | 2-pentene |
| UiO-67 | 98.344 | 0 | 0 | 0.013 | 0 |
| EMM-35 | 57.894 | 1.058 | 0.041 | 1.719 | 2.668 |
| EMM-23 (Al) | 50.314 | 4.486 | 0.208 | 7.693 | 0.653 |

Interestingly, both EMM-35 and EMM-23 appear to have a substantial yield of methyl-shift reaction products while EMM-35 appears to produce a substantial yield, in particular, of dimethyl product.

The following methods were employed to characterize the MOF and zeolitic isomerization catalysts described herein:

Powder X-Ray Diffraction (PXRD) patterns were collected with a PANalytical X-Pert Pro diffraction system, equipped with an X′Celerator detector, using copper K-alpha radiation. The diffraction data were recorded by step-scanning at 0.017 degrees of two-theta, where theta is the Bragg angle, and a counting time of 21 seconds for each step. The interplanar spacings, d-spacings, were calculated in angstrom (Å) units and relative peak area intensities were determined with the MDI Jade peak profile-fitting algorithm.

NMR data were recorded on Bruker 400 MHz and 500 MHz NMR Spectrometers. ¹⁹F NMR chemical shifts are reported relative to trifluoroacetic acid at δ=−76.55 ppm. Attenuated total reflectance (ATR) Fourier transform infrared (FTIR) data were recorded on a Bruker Alpha IR instrument using a single-bounce Diamond ATR crystal.

As used in the present disclosure and claims, the singular forms “a,” “an,” and “the” include plural forms unless the context clearly dictates otherwise. The term “and/or” as used in a phrase such as “A and/or B” herein is intended to include “A and B”, “A or B,” “A,” and “B.” Further, while compositions and methods are described herein in terms of “comprising” various components or steps, the compositions and methods may also “consist essentially of” or “consist of” the various components and steps.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present disclosure. At the very least, and not as an attempt to limit the application of the doctrine of equivalents to the scope of the claim, each numerical parameter should at least be construed in light of the number of reported significant digits and by applying ordinary rounding techniques.

All documents described herein are incorporated by reference herein for purposes of all jurisdictions where such practice is allowed, including any priority documents and/or testing procedures to the extent that they are not inconsistent with this text.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Further, not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for one of ordinary skill in the art and having benefit of this disclosure.

We claim:

1. A method for converting an olefin comprising:
contacting a feed comprising the olefin with a catalyst comprising metal organic framework (MOF) under conditions effective to generate a product comprising a branched isomer of the olefin, wherein not more than about 10 wt. % of the product comprises a cracking product or a molecular-weight growth reaction product, wherein a weight ratio of branched olefins in the product to molecular-weight (MW) growth to reaction product in the product is greater than about 4:
$$\frac{wt . branched olefins}{wt . MW growth product} > 4.$$

2. The method of claim 1, wherein the MOF is characterized by a plurality of Zr₆O₄(OH)₄ octahedra twelve-fold bonded together by a plurality of 4,4′-biphenyldicarboxylate linking ligands and, on average, at least one perhalo-4,4′-biphenylbisphosphonate linking ligand.

3. The method as in claim 2, wherein the perhalo-4,4′-biphenylbisphosphonate linking ligand is perfluoro-4,4′-biphenylbisphosphonate linking ligand.

4. The method as in claim 1, wherein the metal organic framework is EMM-35.

5. The method as in claim 2, wherein the ratio of 4,4′-biphenyldicarboxylate linking ligands to perhalo-4,4′-biphenylbisphosphonate linking ligand is from about 3:2 to about 2:3.

6. The method as in claim 1, wherein the catalyst further comprises a binder.

7. A method for converting an olefin comprising:
contacting a feed comprising the olefin with a catalyst comprising a zeolite under conditions effective to generate a product comprising a branched isomer of the olefin, wherein not more than about 10 wt. % of the product comprises a cracking product or a molecular-weight growth reaction product, and wherein a weight ratio of branched olefins in the product to molecular-weight (MW) growth reaction product in the product is greater than about 4:
$$\frac{wt . branched olefins}{wt . MW growth product} > 4.$$

8. The method as in claim 7, wherein the catalyst comprises a zeolite comprising pores defined by rings of 14 or more tetrahedral atoms.

9. The method as in claim 7, wherein the zeolite comprises pores defined by rings of 21 or more tetrahedral atoms.

10. The method as in claim 7, wherein the zeolite is characterized by an empirical chemical formula of (X₂O₃)ₘ(YO₂)ₙ, wherein m is about 10 or greater, n is 0 or a positive integer, X is a trivalent element selected from one or more of B, Al, Fe, and Ga, and Y is a tetravalent element selected from one or more of Si, Ge, Sn, Ti, and Zr.

11. The method as in claim 7, wherein the zeolite comprises one or both of framework alumina and extraframework alumina.

12. The method as in claim 7, wherein the catalyst comprises one or more impurities selected from the group consisting of ZSM-5 zeolites, beta zeolites, sponge-like morphologies, quartz, tridymite, analcite, clathrate, and amorphous materials.

13. The method as in claim 7, wherein the catalyst further comprises a binder.

14. The method as in claim 1, wherein the research octane number (RON) of the product is higher than the RON of the feed.

15. The method as in claim 1, wherein the feed comprises one or both of coker naphtha and cracked naphtha.

16. The method as in claim 1, wherein the feed further comprises one or both of a paraffin and an aromatic hydrocarbon.

17. The method as in claim 1, wherein the product comprises a higher weight percentage of branched olefins than the feed.

18. The method as in claim 1, wherein the conditions effective comprise a temperature of about 150° C. to about 300° C.

19. The method as in claim 1, wherein the conditions effective comprise a pressure range of about 100 psig (689 KPa) to about 1000 psig (6.89 MPa).

20. The method as in claim 1, wherein the conditions effective comprise a pressure range of about 200 psig (about 1.38 MPa) to about 300 psig (about 2.07 MPa).

21. The method as in claim 1, wherein at least a fraction of the product is isolated and combined with the feed.

22. The method as in claim 1, further comprising subjecting at least a portion of the product to hydrotreating conditions thereby forming a hydrotreated product having a higher octane than a product formed from subjecting the feed to the same hydrotreating conditions.
